(19) United States
(12) Patent Application Publication
(10) Pub. No.: US 2026/0199482 A1
(43) Pub. Date: Jul. 16, 2026

(54) BILE ACID-METRONIDAZOLE CONJUGATE AND USE THEREOF

(71) Applicant: SOUTH CHINA UNIVERSITY OF TECHNOLOGY, Guangzhou, Guangdong (CN)

(72) Inventors: Lei Zhang, Guangzhou, Guangdong (CN); Fan Zhong, Guangzhou, Guangdong (CN); Kun Shi, Guangzhou, Guangdong (CN); Shan Li, Guangzhou, Guangdong (CN); Jing Li, Guangzhou, Guangdong (CN)

(21) Appl. No.: 19/137,285

(22) PCT Filed: Nov. 21, 2023
(86) PCT No.: PCT/CN2023/132991

§ 371 Date: Jun. 9, 2025

(30) Foreign Application Priority Data
Dec. 9, 2022 (CN) 202211578060.2

(51) Int. Cl. A61K 47/54; A61K 47/55; A61P 31/04

(52) U.S. Cl. CPC A61K 47/545; A61K 47/552; A61K 47/554; A61P 31/04

(57) ABSTRACT

Disclosed is a bile acid-metronidazole conjugate or a stereoisomer thereof, or a salt thereof, or a solvate thereof, or a prodrug thereof, or a metabolite thereof. The bile acid-metronidazole conjugate is prepared using an esterification reaction of a bile acid compound with hydroxyl group on a metronidazole structure. The synthesis process is simple and easy for industrialization. The bile acid-metronidazole conjugate can effectively inhibit the growth of anaerobic bacteria, especially the growth of Clostridioides difficile and Helicobacter pylori, so that the bile acid-metronidazole conjugate has a significantly improved therapeutic effect in the treatment of infections caused by the anaerobic bacteria.

### TECHNICAL FIELD

The present application belongs to the field of medicinal technology, and more particularly, relates to a bile acid-metronidazole conjugate and use thereof.

### BACKGROUND ART

Nidazole compounds are a class of antibiotics that can be used to treat or prevent systemic or local infections caused by anaerobic bacteria, mainly including metronidazole, ornidazole, tinidazole and secnidazole, etc., which have been widely used in clinical practice. As a first-line drug for clinical practice, the dosage of metronidazole for adults is relatively large (about 1.2-2.5 g/d), and about 15% to 30% cases will experience adverse reactions, such as nausea, vomiting, headache, and dizziness, and in severe cases, convulsions, etc. The adverse reactions will gradually return to normal after discontinuation of medication. Patients with impaired liver or kidney functions need to have the dosage reduced. In order to improve the clinical use of metronidazole, Galculus Bovis and Metronidazole Compound Preparation has been developed, which are applied in acute pericoronitis of wisdom tooth, pulpitis, periapical periodontitis, etc. Metronidazole has also been developed into prodrug forms, such as metronidazole benzoate, metronidazole phosphate or disodium salts thereof, metronidazole acetate, metronidazole butyrate, metronidazole oleate, metronidazole palmitate, etc. to improve the pharmaceutical characteristics and pharmacodynamic characteristics of metronidazole.

Bile acid compounds are a class of tetracyclic fused ring steroid compounds with similar structures and generally containing 24 carbon atoms. They have the effects of regulating intestinal flora and resisting infections. In recent years, there have been literatures indicating that secondary bile acids have a certain regulatory effect on the growth of Clostridioides difficile (Y. Sato, K. Atarashi, D. R. Plichta, et al, Novel bile acid biosynthetic pathways are enriched in the microbiome of centenarians, Nature, 2021, 599, 458-464; B. H. Mullish, J. R. Allegretti, The contribution of bile acid metabolism to the pathogenesis of Clostridioides difficile infection, Therapeutic Advances in Gastroenterology, 2021, 14, 1-12.). For instance, ursodeoxycholic acid (UDCA) can alleviate inflammations by altering gut bile acids (J. A. Winston, A. J. Rivera, J. Cai, et al, Ursodeoxycholic acid (UDCA) mitigates the host inflammatory response during Clostridioides difficile infection by altering gut bile acids, Infection and Immunity, 2020, 88, e00045-20).

Metronidazole is used for abdominal complex infections with its good pharmacodynamic characteristics, e.g., for the treatment of gastric ulcers caused by Helicobacter pylori infections in conjunction with other drugs. Metronidazole was also recommended by the American Gastroenterology Association for the treatment of patients with first episode of mild and moderate Clostridioides difficile infections. Although antibiotics can be used to treat acute infections, they can also cause imbalance and deterioration of intestinal flora, making patients more prone to recurrent infections and leading to a vicious cycle. Considering that the bacteria's resistance to drugs will gradually increase during the evolution process, and the emergence of refractory strains that are resistant to metronidazole and vancomycin have been observed in clinical practice, so it is urgent to develop new antibacterial agents with high activity that could inhibit Clostridioides difficile and Helicobacter pylori infections.

### SUMMARY OF THE INVENTION

In view of the above technical defects, the examples of the present application provide a novel bile acid-metronidazole conjugate, which is intended to prepare a novel antibiotic drug to enhance the efficacy of metronidazole, for the treatment or inhibition of anaerobic bacteria infection (including infections of Clostridioides difficile, Helicobacter pylori, etc.).

An example of the present application discloses a bile acid-metronidazole conjugate having a chemical structure as shown in formula (I), or a stereoisomer thereof, or a salt thereof, or a solvate thereof, or a prodrug thereof, or a metabolite thereof;

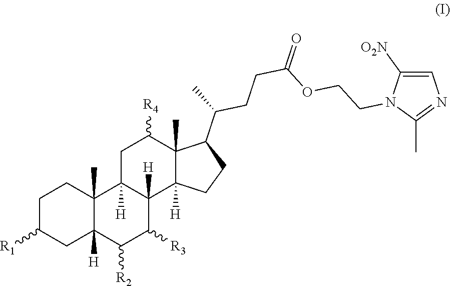

wherein, ~~ represents a single bond or double bonds, and ~~R1, ~~R2, ~~R3, ~~R4 each represents one of —H, —OH, and ═O.

The conjugate, or the stereoisomer thereof, or the salt thereof, or the solvate thereof, or the prodrug thereof, or the metabolite thereof is selected from the following structures:

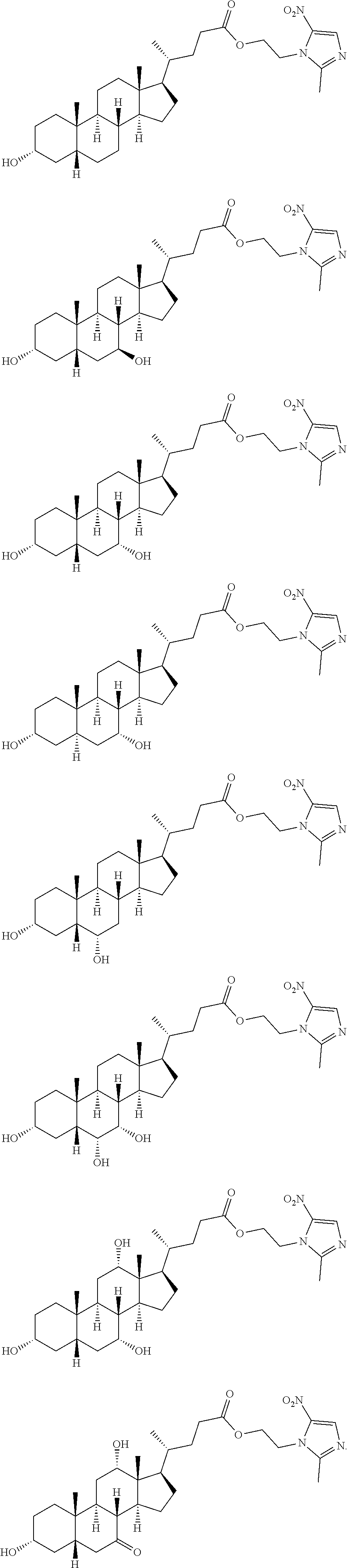

The bile acid-metronidazole conjugate is prepared by an esterification reaction of a bile acid compound with metronidazole.

The bile acid compound is selected from one of cholic acid, ursodeoxycholic acid, chenodeoxycholic acid, allo-chenodeoxycholic acid, hyodeoxycholic acid, hyocholic acid, lithocholic acid and derivatives thereof.

Disclosed is use of a bile acid-metronidazole conjugate, i.e., use of the bile acid-metronidazole conjugate in the inhibition of the growth of anaerobic bacteria.

The anaerobic bacteria include, but are not limited to, Clostridioides difficile, Bacteroides fragilis, and Porphyromonas gingivalis.

The present application provides a bile acid-metronidazole conjugate, a stereoisomer thereof, or a salt thereof, or a solvate thereof, or a prodrug thereof, or a metabolite thereof. The bile acid-metronidazole conjugate is prepared using an esterification reaction of one of bile acid compounds with hydroxyl group on a metronidazole structure. The synthesis process is simple and easy for industrialization. The bile acid-metronidazole conjugate can effectively inhibit the growth of anaerobic bacteria, especially the growth of Clostridioides difficile, so that the bile acid-metronidazole conjugate has a significantly improved therapeutic effect in the treatment of infections caused by the anaerobic bacteria.

### BRIEF DESCRIPTION OF THE DRAWINGS

FIG. 1 is a mouse experimental flowchart in a Clostridioides difficile infection (CDI) model;

FIG. 2 is a tendency chart of changes in mouse body weights; and

FIG. 3 is a graph of mouse survival curves.

### DETAILED DESCRIPTION OF THE INVENTION

In order to make the objectives, technical solutions and advantages of the examples in the present application more clearly, the technical solutions in the examples of the present application will be described clearly and completely in conjunction with the accompanying drawings in the examples of the present application. Apparently, the described examples are merely some examples, rather than all examples, of the present application. Based on the examples in the present application, all other examples derived by a person of ordinary skill in the art without creative efforts shall fall within the protection scope of the present application.

Various raw materials in the examples of the present application are introduced as follows:
Hydrochloric acid, purchased from Sinopharm Chemical Reagents Co., Ltd., with CAS number 7647-01-0;
Sodium bicarbonate, purchased from Sinopharm Chemical Reagents Co., Ltd., with CAS number 144-55-8;
Anhydrous sodium sulfate, purchased from Sinopharm Chemical Reagents Co., Ltd., with CAS number 7757-82-6;
Lithocholic acid, purchased from Zhongshan Bailing Biotechnology Co., Ltd., with CAS number 434-13-9;
Metronidazole, purchased from Shanghai Aladdin Biochemical Technology Co., Ltd., with CAS number 443-48-1;
1-Ethyl-3-(3-dimethylaminopropyl)carbodiimide hydrochloride, purchased from Shanghai Aladdin Biochemical Technology Co., Ltd., with CAS number 25952-53-8;
Dimethylaminopyridine, purchased from Shanghai Aladdin Biochemical Technology Co., Ltd., with CAS number 5683-33-0;
Dichloromethane, purchased from Sinopharm Chemical Reagents Co., Ltd., with CAS number 75-09-2;
Petroleum ether, purchased from Sinopharm Chemical Reagents Co., Ltd., with CAS number 8032-32-4;
Ethyl acetate, purchased from Sinopharm Chemical Reagents Co., Ltd., with CAS number 141-78-6;
Ursodeoxycholic acid, purchased from Zhongshan Bailing Biotechnology Co., Ltd., with CAS number 128-13-2;
Chenodeoxycholic acid, purchased from Zhongshan Bailing Biotechnology Co., Ltd., with CAS number 474-25-9;
Allo-chenodeoxycholic acid, purchased from Zhongshan Bailing Biotechnology Co., Ltd., with CAS number 15357-34-3;
Hyodeoxycholic acid, purchased from Zhongshan Bailing Biotechnology Co., Ltd., with CAS number 83-49-8;
Hyocholic acid, purchased from Zhongshan Bailing Biotechnology Co., Ltd., with CAS number 547-75-1; and
Cholic acid, purchased from Zhongshan Bailing Biotechnology Co., Ltd., with CAS number 81-25-4.

It should also be understood that when the terms, “include/comprise” and “contain” used in the present specification and attached claims mean a described feature, integer, step, operation, element and/or component, it could not preclude existence or addition of one or more other features, integers, steps, operations, elements, components and/or combinations thereof.

It should also be understood that the terms used in the specification of the present application are for the purpose of describing particular examples only and are not intended to limit the present application. The singular forms “a/an”, “one” and “the” as used in the specification of the present application and the attached claims are intended to include the plural forms as well, unless the context clearly indicates otherwise.

It should also be further understood that the term “and/or” as used in the specification of the present application and the attached claims refers to any combination of one or more of the associated listed items and all possible combinations, and includes these combinations.

Examples of the present application disclose a bile acid-metronidazole conjugate having a chemical structure as shown in formula (I), or a stereoisomer thereof, or a salt thereof, or a solvate thereof, or a prodrug thereof, or a metabolite thereof;

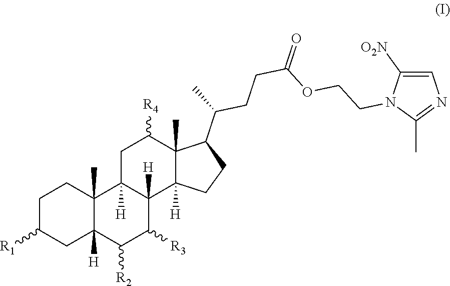

wherein, [symbol] represents a single bond or double bonds, and ~~R1, ~~R2, ~~R3, ~~R4 each represents one of —H, —OH, and ═O.

The bile acid-metronidazole conjugate, or the stereoisomer thereof, or the salt thereof, or the solvate thereof, or the prodrug thereof, or the metabolite thereof is selected from the following structures:

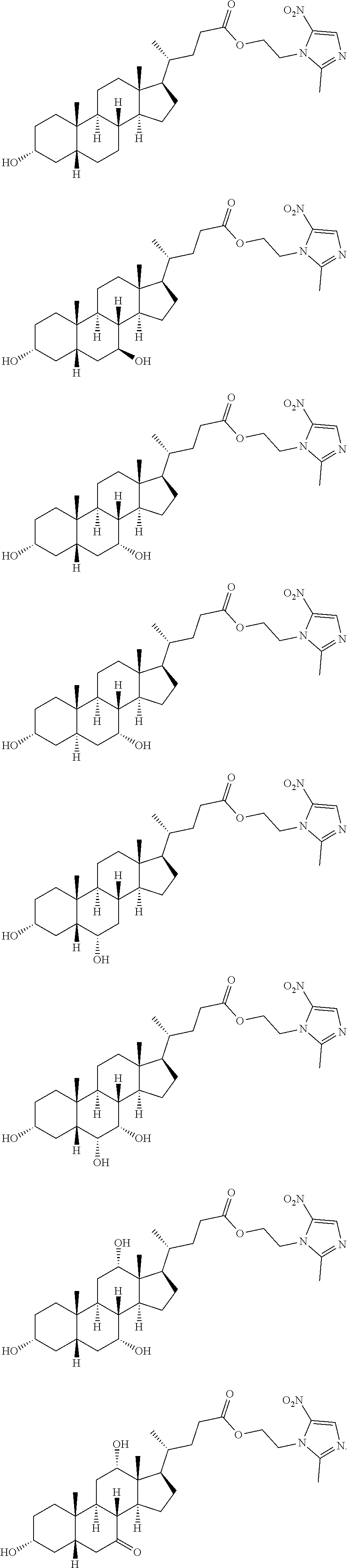

the bile acid compound is selected from one of cholic acid, ursodeoxycholic acid, chenodeoxycholic acid, allo-chenodeoxycholic acid, hyodeoxycholic acid, lithocholic acid and derivatives thereof.

Disclosed is usage of a bile acid-metronidazole conjugate, i.e., usage of the bile acid-metronidazole conjugate in inhibiting the growth of anaerobic bacteria.

The anaerobic bacteria include, but are not limited to, Clostridioides difficile, Bacteroides fragilis, and Porphyromonas gingivalis.

The bile acid-metronidazole conjugate can also prevent Clostridioides difficile and Helicobacter pylori infectious diseases, the recurrence of Clostridioides difficile infectious diseases, or complications of Clostridioides difficile infectious diseases.

### Example 1: Synthesis of Compound SCUT1-1

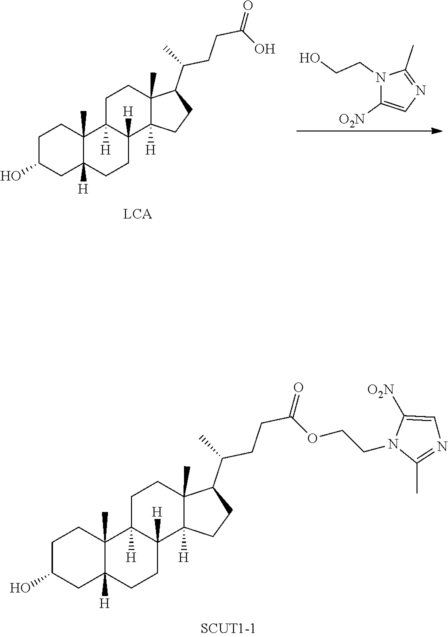

1.01 g of lithocholic acid (LCA), 0.54 g of metronidazole, 0.75 g of 1-ethyl-3-(3-dimethyl-aminopropyl)carbodiimide hydrochloride, 0.48 g of dimethylaminopyridine and 50 mL of dichloromethane were added to a 100 mL single-mouth flask, and stirred at room temperature for 5-6 h; the reaction progress was monitored by TLC until the LCA reaction was completed; the reaction solution was poured into a 250 mL separatory funnel, and added with 50 mL of water and 10 mL of dilute hydrochloric acid (1 M), and an organic phase was separated by extraction; 50 mL of water and 10 mL of dilute hydrochloric acid (1 M) were added to the organic phase, and an organic phase was separated by washing; 100 mL of saturated sodium bicarbonate solution was added to the organic phase, and an organic phase was separated by washing; anhydrous sodium sulfate (5.0 g) was added to the organic phase for drying, and the solid was removed by filtration; the filtrate was concentrated in vacuum; the residue was subject to silica gel column chromatography, and a petroleum ether/ethyl acetate (v/v=1:3) system was used as an eluent; and after the solvent was removed by evaporation, 0.81 g of white solid was obtained.

¹H NMR (600 MHz, CDCl₃) δ 7.96 (s, 1H), 4.59 (t, J=5.3 Hz, 2H), 4.41 (m, 2H), 3.62 (m, 1H), 2.52 (s, 3H), 2.30 (ddd, J=15.6, 10.4, 5.2 Hz, 1H), 2.17 (ddd, J=15.9, 10.0, 6.4 Hz, 1H), 1.94 (dt, J=12.3, 3.2 Hz, 1H), 1.88-1.62 (m, 8H), 1.60-1.48 (m, 2H), 1.44-1.37 (m, 5H), 1.37-1.31 (m, 2H), 1.31-1.18 (m, 5H), 1.13 (td, J=12.8, 3.6 Hz, 1H), 1.10-1.00 (m, 4H), 0.97 (td, J=14.2, 3.5 Hz, 1H), 0.92 (s, 3H), 0.88 (d, J=6.5 Hz, 3H), 0.63 (s, 3H);

¹³C NMR (151 MHz, CDCl₃) δ 173.61, 150.80, 138.55, 133.13, 71.85, 62.36, 56.47, 55.82, 45.09, 42.74, 42.08, 40.42, 40.14, 36.46, 35.84, 35.35, 35.29, 34.57, 30.93, 30.78, 30.55, 28.19, 27.18, 26.41, 24.19, 23.37, 20.81, 18.24, 14.39, 12.04;

HRMS-ESI(m/z): C₃₀H₄₇N₃O₅ measured value: 530.3581 [M+H]⁺, 552.3405 [M+Na]⁺.

### Example 2: Synthesis of Compound SCUT1-2

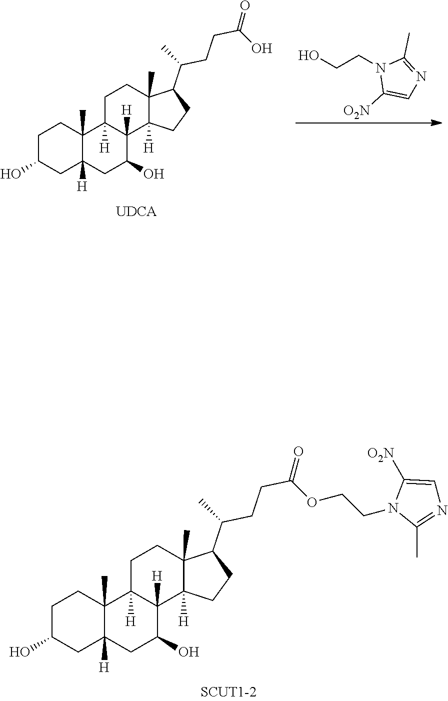

SCUT1-2 was synthesized by the same method as Example 1 using ursodeoxycholic acid (UDCA) instead of LCA as a reactant.

¹H NMR (600 MHz, CDCl₃) δ 7.96 (s, 1H), 4.59 (t, J=5.3 Hz, 2H), 4.45-4.36 (m, 2H), 3.59 (m, 2H), 2.52 (s, 3H), 2.31 (ddd, J=15.6, 10.3, 5.2 Hz, 1H), 2.17 (ddd, J=16.0, 10.0, 6.3 Hz, 1H), 1.98 (dt, J=12.5, 3.3 Hz, 1H), 1.91-1.76 (m, 5H), 1.73 (m, 2H), 1.69-1.65 (m, 3H), 1.62-1.55 (m, 3H), 1.52-1.35 (m, 7H), 1.32-1.20 (m, 6H), 1.14 (td, J=12.9, 3.9 Hz, 1H), 1.07-0.99 (m, 2H), 0.95 (s, 3H), 0.90 (d, J=6.5 Hz, 3H), 0.66 (s, 3H);

¹³C NMR (151 MHz, CDCl₃) δ 173.55, 150.78, 133.09, 71.45, 71.37, 62.37, 55.69, 54.78, 45.07, 43.78, 43.76, 42.44, 40.10, 39.17, 37.30, 36.88, 35.18, 34.93, 34.08, 30.92, 30.79, 30.35, 28.59, 26.87, 23.38, 21.16, 18.36, 14.38, 12.13;

HRMS-ESI(m/z): C₃₀H₄₇N₃O₆ measured value: 546.3535 [M+H]⁺, 568.3352 [M+Na]⁺.

### Example 3: Synthesis of Compound SCUT1-3

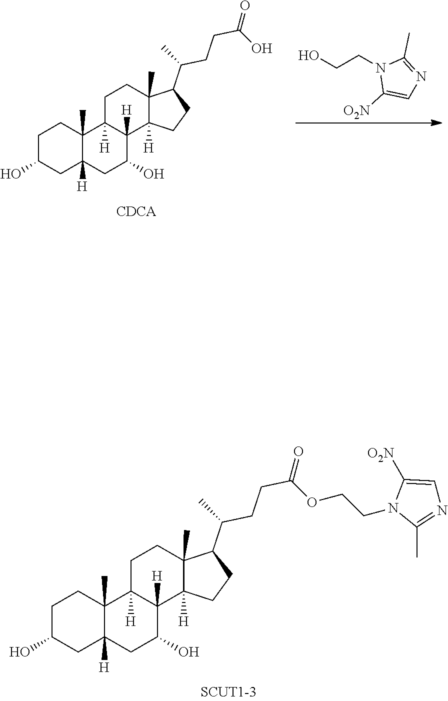

SCUT1-3 was synthesized by the same method as Example 1 using chenodeoxycholic acid (CDCA) instead of LCA as a reactant.

¹H NMR (600 MHz, CDCl₃) δ 7.96 (s, 1H), 4.59 (t, J=5.3 Hz, 2H), 4.41 (td, J=5.3, 2.9 Hz, 2H), 3.85 (q, J=3.1 Hz, 1H), 3.47 (tt, J=10.9, 4.4 Hz, 1H), 2.52 (s, 3H), 2.30 (ddd, J=15.5, 10.2, 5.1 Hz, 1H), 2.23-2.15 (m, 2H), 1.97 (m, 3H), 1.84 (m, 4H), 1.75-1.60 (m, 8H), 1.50-1.48 (m, 2H), 1.42-1.25 (m, 12H), 1.15 (m, 4H), 0.98 (td, J=14.2, 3.2 Hz, 2H), 0.90 (d, J=7.3 Hz, 6H), 0.65 (s, 3H);

¹³C NMR (151 MHz, CDCl₃) δ 173.57, 150.79, 133.13, 72.02, 68.53, 62.38, 55.61, 50.45, 45.09, 42.70, 41.47, 39.91, 39.60, 39.42, 35.32, 35.27, 35.05, 34.63, 32.83, 30.83, 30.74, 30.68, 29.71, 28.15, 23.70, 22.77, 20.57, 18.24, 14.41, 11.78;

HRMS-ESI(m/z): C₃₀H₄₇N₃O₆ measured value: 546.3536 [M+H]⁺, 568.3350 [M+Na]⁺.

### Example 4: Synthesis of Compound SCUT1-4

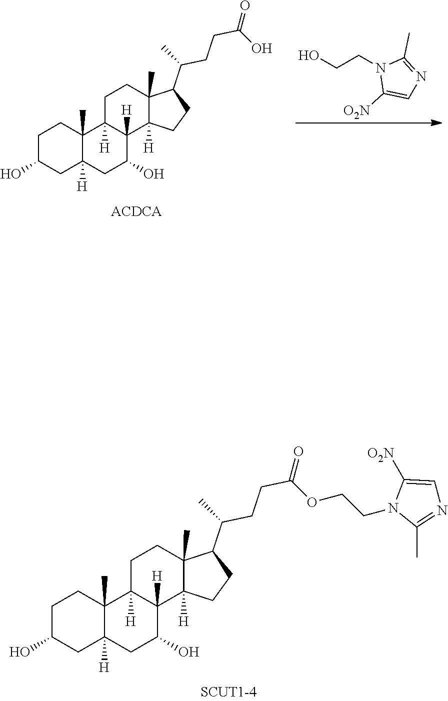

SCUT1-4 was synthesized by the same method as Example 1 using allo-chenodeoxycholic acid (ACDCA) instead of LCA as a reactant.

¹H NMR (600 MHz, CDCl₃) δ 7.96 (s, 1H), 4.59 (t, J=5.3 Hz, 2H), 4.41 (td, J=5.5, 2.8 Hz, 2H), 4.05(q, J=2.9 Hz, 1H), 3.83 (t, J=3.0 Hz, 1H), 2.52 (s, 3H), 2.30 (ddd, J=15.6, 10.3, 5.2 Hz, 1H), 2.17 (ddd, J=15.9, 9.9, 6.4 Hz, 1H), 2.04 (tt, J=13.0, 3.5 Hz, 1H), 1.92 (dt, J=12.9, 3.2 Hz, 1H), 1.84 (m, 2H), 1.76-1.61 (m, 8H), 1.59-1.48 (m, 4H), 1.48-1.41 (m, 3H), 1.37 (m, 6H), 1.28 (m, 7H), 1.11 (m, 4H), 0.89 (d, J=6.5 Hz, 3H), 0.78 (s, 3H), 0.64 (s, 3H);

¹³C NMR (151 MHz, CDCl₃) δ 173.59, 150.81, 138.47, 133.14, 67.99, 66.44, 62.38, 55.58, 50.62, 45.87, 45.09, 42.65, 39.50, 39.45, 36.30, 36.19, 35.58, 35.27, 31.95, 31.59, 30.83, 30.74, 28.85, 28.07, 23.56, 20.53, 18.23, 14.40, 11.86, 10.13;

HRMS-ESI(m/z): C₃₀H₄₇N₃O₆ measured value: 546.3532 [M+H]⁺, 568.3356 [M+Na]⁺.

### Example 5: Synthesis of Compound SCUT1-5

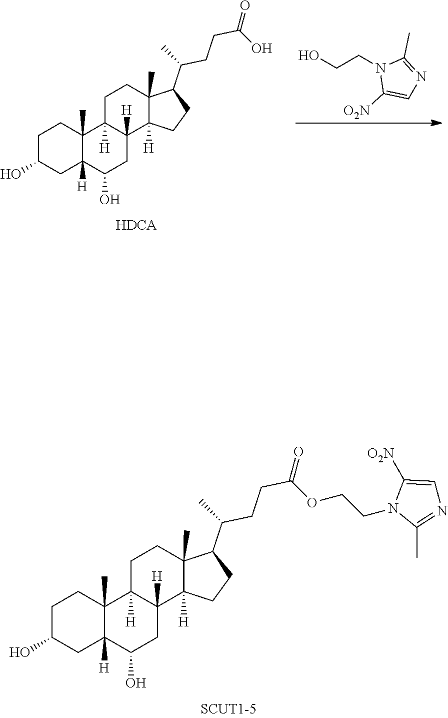

SCUT1-5 was synthesized by the same method as Example 1 using hyodeoxycholic acid (HDCA) instead of LCA as a reactant.

¹H NMR (600 MHz, CDCl₃) δ 7.96 (s, 1H), 4.59 (t, J=5.3 Hz, 2H), 4.41 (q, J=5.1 Hz, 2H), 4.05 (dt, J=12.1, 4.8 Hz, 1H), 3.62 (tt, J=10.7, 4.6 Hz, 1H), 2.52 (s, 3H), 2.31 (ddd, J=15.6, 10.3, 5.2 Hz, 1H), 2.17 (ddd, J=16.0, 10.0, 6.4 Hz, 1H), 1.98-1.92 (m, 2H), 1.87-1.75 (m, 3H), 1.75-1.55 (m, 6H), 1.48-1.02 (m, 18H), 0.90 (s, 3H), 0.88 (d, J=6.5 Hz, 3H), 0.63 (s, 3H);

¹³C NMR (151 MHz, CDCl₃) δ 173.57, 150.80, 138.55, 133.05, 71.57, 68.04, 62.37, 56.11, 55.78, 48.39, 45.08, 42.85, 39.91, 39.80, 35.95, 35.56, 35.26, 35.00, 34.82, 30.91, 30.72, 30.23, 29.19, 28.10, 24.17, 23.48, 20.74, 18.22, 14.36, 12.02;

HRMS-ESI(m/z): C₃₀H₄₇N₃O₆ measured value: 546.3529 [M+H]⁺, 568.3361 [M+Na]⁺.

### Example 6: Synthesis of Compound SCUT1-6

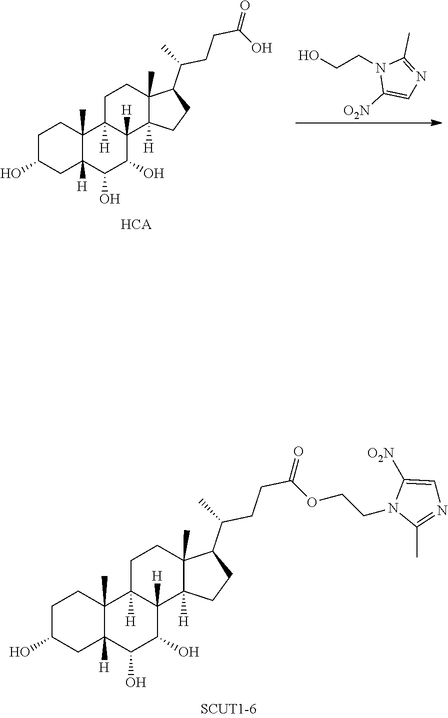

SCUT1-6 was synthesized by the same method as Example 1 using hyocholic acid (HCA) instead of LCA as a reactant.

¹H NMR (600 MHz, MeOH-d₄) δ 7.93 (s, 1H), 4.67 (t, J=5.2 Hz, 2H), 4.47-4.39 (m, 2H), 3.79-3.75 (m, 2H), 3.37-3.32 (m, 1H), 2.52 (s, 3H), 2.31 (ddd, J=15.2, 9.7, 5.3 Hz, 1H), 2.18 (ddd, J=15.9, 9.5, 6.9 Hz, 1H), 1.98 (td, J=7.0, 4.1 Hz, 2H), 1.95-1.66 (m, 7H), 1.62 (dt, J=12.1, 3.3 Hz, 1H), 1.58-1.47 (m, 4H), 1.39-1.00 (m, 12H), 0.92 (s, 3H), 0.90 (d, J=6.6 Hz, 3H), 0.66 (s, 3H);

¹³C NMR (151 MHz, MeOH-d₄) δ 175.10, 152.59, 132.68, 72.94, 72.84, 70.77, 63.44, 57.21, 51.40, 49.57, 46.25, 43.79, 40.95, 40.09, 37.07, 36.85, 36.68, 33.97, 33.37, 32.08, 31.80, 31.43, 29.21, 24.55, 23.73, 21.81, 18.76, 14.14, 12.20;

HRMS-ESI(m/z): C₃₀H₄₇N₃O₇ measured value: 584.3308 [M+Na]⁺.

### Example 7: Synthesis of Compound SCUT1-7

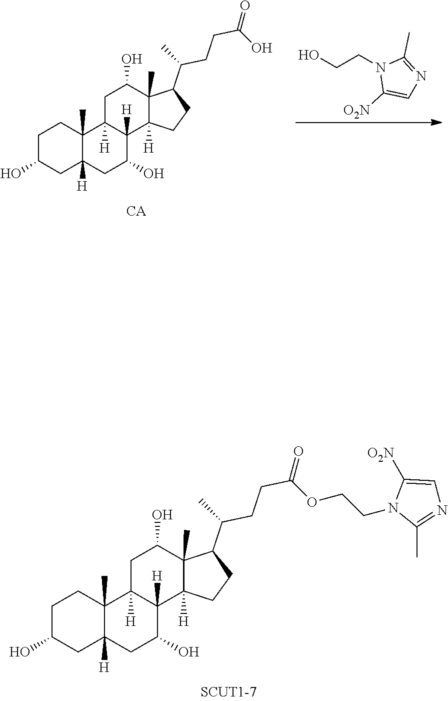

SCUT1-7 was synthesized by the same method as Example 1 using cholic acid (CA) instead of LCA as a reactant.

¹H NMR (600 MHz, MeOH-d₄) δ 7.84 (s, 1H), 4.57 (t, J=5.2 Hz, 2H), 4.33 (m, 2H), 3.83 (d, J=3.0 Hz, 1H), 3.70 (q, J=3.0 Hz, 1H), 3.27 (tt, J=11.2, 4.4 Hz, 1H), 2.42 (s, 3H), 2.16-2.06 (m, 2H), 1.92-1.83 (m, 2H), 1.77-1.73 (m, 1H), 1.71 (dt, J=13.7, 3.4 Hz, 2H), 1.66-1.57 (m, 2H), 1.51-1.41 (m, 5H), 1.37-1.12 (m, 7H), 1.00 (m, 1H), 0.86 (d, J=6.5 Hz, 3H), 0.82 (s, 3H), 0.59 (s, 3H);

¹³C NMR (151 MHz, MeOH-d₄) δ 173.72, 151.16, 131.23, 72.56, 71.48, 67.63, 61.98, 46.47, 46.07, 44.83, 41.81, 41.58, 39.62, 39.07, 35.21, 35.09, 34.50, 34.44, 30.63, 30.31, 29.78, 28.18, 27.22, 26.48, 22.79, 21.75, 16.15, 12.70, 11.56;

HRMS-ESI(m/z): C₃₀H₄₇N₃O₇ measured value: 584.3304 [M+Na]⁺.

### Example 8: Synthesis of Compound SCUT1-8

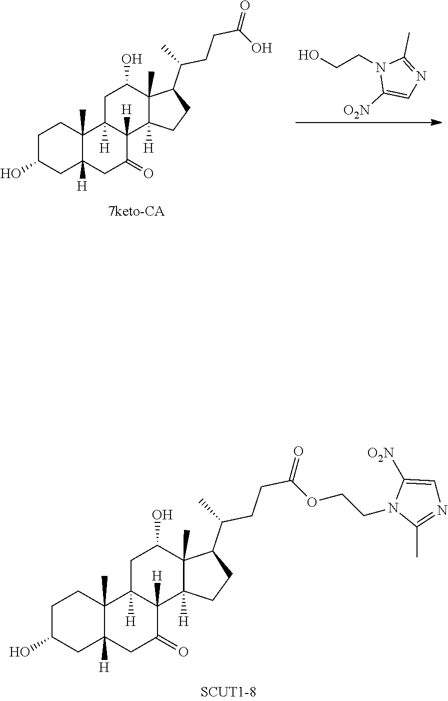

SCUT1-8 was synthesized by the same method as Example 1 using 7-ketolithocholic acid (7keto: CA) instead of LCA as a reactant.

¹H NMR (600 MHz, CDCl₃) δ 8.05-8.01 (m, 1H), 4.68-4.58 (m, 2H), 4.43 (m, 2H), 3.97 (d, J=3.0 Hz, 1H), 3.59 (td, J=10.9, 5.3 Hz, 1H), 2.83 (dd, J=12.6, 6.0 Hz, 1H), 2.59 (s, 3H), 2.43-2.06 (m, 11H), 1.98-1.95 (m, 1H), 1.95-1.89 (m, 2H), 1.89-1.86 (m, 1H), 1.86-1.73 (m, 3H), 1.74-1.62 (m, 5H), 1.56 (dt, J=14.3, 3.7 Hz, 1H), 1.40-1.13 (m, 17H), 1.00-0.83 (m, 6H), 0.66 (s, 3H);

¹³C NMR (151 MHz, CDCl₃) δ 211.57, 173.51, 131.80, 107.14, 71.95, 70.89, 62.30, 49.51, 46.49, 46.05, 45.36, 40.69, 37.42, 35.98, 34.84, 34.71, 34.12, 30.66, 30.55, 29.79, 29.71, 29.31, 27.60, 24.26, 22.82, 17.35, 14.19, 12.82;

HRMS-ESI(m/z): C₃₀H₄₅N₃O₇ measured value: 560.3326 [M+H]⁺, 582.3141 [M+Na]⁺.

The following experimental examples were used to prove the beneficial effects of the compounds of the present application.

### Example 1: In-Vitro Inhibitory Activities of Compounds of the Present Application on Anaerobic Bacteria

### (I) Experimental Method

Experimental strains: Clostridioides difficile ATCC 43255, Bacteroides fragilis ATCC 25285, and Porphyromonas gingivalis ATCC 33277.

### Experimental Process:

S1, a cycloserine-cefoxitin-fructose agar medium, a tryptone soy broth medium, a brain heart infusion broth medium, a fortified Brucella broth medium, a Colombian blood agar plate, and an anaerobic blood agar plate were produced or prepared, respectively.

The cycloserine-cefoxitin-fructose agar medium was prepared as follows: 53.2 g of cycloserine-cefoxitin-fructose agar medium was weighed, dissolved in 1 L of distilled water by heating, aliquoted in conical flasks, and autoclaved at 121° C. for 15 min. 0.10 g of sterile cycloserine and 3.20 mg of sterile cefoxitin (filtered by a filter membrane and sterilized) were added to every 200 mL, mixed well, and poured into a sterile petri dish for later use.

The tryptone soy broth medium was prepared as follows: 30.0 g of dry powder of the tryptone soy broth medium was weighed, dissolved in 1 L of distilled water by heating, aliquoted in conical flasks, and autoclaved at 121° C. for 15 min.

The brain heart infusion broth medium was prepared as follows: 37.0 g of brain heart infusion broth medium was weighed, dissolved in 1 L of distilled water, aliquoted in conical flasks, and autoclaved at 121° C. for 15 min.

The fortified Brucella broth medium was prepared as follows: a 5 mg/mL chlorhematin solution, a 4 mg/L sodium hydroxide solution and a vitamin K1 solution were prepared, respectively; 28.1 g of Brucella broth dry powder medium was weighed, dissolved in 900 mL of distilled water, added with 28.0 g of Brucella agar powder, 1 mL of chlorhematin solution and 1 mL of vitamin K1 working solution, and autoclaved at 121° C. for 15 min. The broth was cooled to 48-50° C., and then added with 100 mL of sterile laked horse blood (50%).

Specifically, a preparation method of the chlorhematin solution (5 mg/mL) was as follows: 0.10 g of chlorhematin was dissolved in 2 mL of NaOH solution (4 mg/L), added with distilled water to 20 mL, autoclaved at 121° C. for 15 min, and stored at 4-8° C. in dark place.

Specifically, a preparation method of sodium hydroxide (4 mg/L) was as follow: 40.0 g of sodium hydroxide was dissolved in 1 L of distilled water and stored at room temperature.

Specifically, a preparation method of the vitamin K1 solution was as follows: 0.20 mL of vitamin K1 was added to 20 mL of 95% ethanol to prepare a 10 mg/mL stock solution, which is then stored at 4-8° C. in a black flask. 1 mL of the stock solution was added in 9 mL of sterilized distilled water to prepare a 1 mg/mL working solution, which is then stored at 4-8° C. in a black flask.

A Columbia blood agar plate and an anaerobic blood agar plate were both purchased from finished-product media.

S2, preparation and dilution of compound mother solution: a bile acid-metronidazole conjugate was dissolved with 100% DMSO as a solvent for preparation, with a mother solution concentration of 1600 g/mL and a test concentration of 32-0.06 g/mL, and diluted with sterile water, in a total of 10 two-fold dilution concentrations.

S3, culture of Clostridioides difficile strain: 1) the strain stored at −80° C. was resuscitated in an anaerobic glove box with a cycloserine-cefoxitin-fructose agar medium plate, and statically cultured at 37° C. in a constant-temperature incubator for 48 h;
2) single colonies were picked, and statically cultured in the brain heart infusion broth medium overnight at 37° C. in the constant-temperature incubator; and
3) the bacterial solution that was statically cultured overnight was transferred into the anaerobic glove box with the brain heart infusion broth medium at a ratio of 1:100, and cultured for 3-4 h until the OD₆₀₀ was between 0.6 and 0.8; and during this process, 5 g/L of yeast extract solution was added to increase a strain concentration.

S4, culture of Bacteroides fragilis strain:
1) the strain stored at −80° C. was resuscitated in the anaerobic glove box with an anaerobic blood agar plate, and statically cultured at 37° C. in the constant-temperature incubator for 48 h; and
2) single colonies were picked, and statically cultured in the tryptone soy broth medium overnight at 37° C., and uniform turbidity appearing in the transparent medium was observed with the naked eyes.

S5, Culture of Porphyromonas gingivalis strain:
the strain stored at −80° C. was resuscitated in the anaerobic glove box with a Colombian blood agar plate, and statically cultured at 37° C. in the constant-temperature incubator for 5-7 days.

Single colonies were picked, and statically cultured at 37° C. in the brain heart infusion broth medium for 2-3 days, and uniform turbidity appearing in the transparent medium was observed with the naked eyes.

The culture process of the strain was operated in the anaerobic glove box. After the operation was completed, the strain was well sealed with an anaerobic bag or an anaerobic box, taken out and then cultured in the constant-temperature incubator.

S6, MIC determination using broth microdilution method:
1) Dilution of bacterial solution: a turbidity meter was used to adjust the turbidity of the bacterial solution to OD₆₀₀=0.1; then the bacterial solution was diluted with a medium at 1:15 to reach a concentration of 1×10⁷ CFU/mL; and 10 μL of bacterial suspension was inoculated into a 96 microplate, such that a final test concentration of bacteria was 1×10⁶ CFU/mL.
2) 0.1 mL of diluted compound solution, i.e., 10 μL of bacterial suspension with adjusted concentration, was inoculated per test well.
3) A control group (100 μL of culture medium+10 μL of bacterial suspension with adjusted concentration) without a drug and a blank control (110 μL of culture medium) were provided on the plate.
4) The microplate that has completed the inoculation was sealed in the anaerobic box and cultured at 37° C. for 48 h; the optical density was determined; the antibacterial activity was calculated; and a drug concentration at which bacteria ceased to grow or grew at the most significant reduction level was calculated as the MIC value.

### (II) Experimental Results:

TABLE 1
The MIC values of the compounds of the present application for
inhibiting the bacterial activity of Clostridioides difficile (in μg/mL)

| | MIC (μg/mL) | | |
|---|---|---|---|
| | Clostridioides | Bacteroides | Porphyromonas |
| | difficile | fragilis | gingivalis |
| Compounds | ATCC 43255 | ATCC 25285 | ATCC 33277 |
| SCUT1-1 | 0.5 | 1 | 2 |
| SCUT1-2 | 0.12 | 1 | 0.25 |
| SCUT1-3 | 0.12 | 4 | 0.5 |
| SCUT1-4 | 2 | 2 | 0.5 |
| SCUT1-5 | 0.25 | 2 | 2 |
| SCUT1-6 | 0.06 | 2 | 0.25 |
| SCUT1-7 | 0.5 | 2 | 0.5 |
| SCUT1-8 | 1 | 1 | 1 |
| Metronidazole | 1-2 | 0.25-2 | 2 |
| UDCA | 16 | >32 | >32 |
| CDCA | 8 | >32 | >32 |
| HCA | 32 | >32 | >32 |

It could be seen from Table 1 that the bile acid-metronidazole conjugate SCUT1-8 showed better antibacterial properties against Clostridioides difficile (ATCC 43255), Bacteroides fragile (ATCC 25285), and Porphyromonas gingivalis (ATCC 33277), wherein the inhibitory effects of the bile acid-metronidazole conjugates SCUT1-2, SCUT1-3, and SCUT1-6 on Clostridioides difficile (ATCC 43255) were particularly prominent. At the same dosage, the inhibitory activities of the bile acid-metronidazole conjugate SCUT1-8 against Clostridioides difficile (ATCC 43255), Bacteroides fragilis (ATCC 25285), and Porphyromonas gingivalis (ATCC 33277) were significantly higher than those of bile acid compounds. At the same dosage, the inhibitory activities of the bile acid-metronidazole conjugate SCUT1-8 against Clostridioides difficile (ATCC 43255), Bacteroides fragilis (ATCC 25285), and Porphyromonas gingivalis (ATCC 33277) were significantly higher than those of metronidazole, wherein the inhibitory activities of some compounds (e.g., SCUT1-6) against Clostridioides difficile have been promoted by more than 50 times, and the inhibitory activities of most compounds against Porphyromonas gingivalis were effectively promoted, which could effectively inhibit the growth of anaerobic bacteria.

### Example 2: In-Vitro Inhibitory Activities of Compounds of the Present Application Against Helicobacter pylori

### (I) Experimental Strains:

Experimental strains: H. pylori ATCC 43504, H. pylori ATCC 26695, H. pylori NCTC 11637, and H. pylori Sydney strain SS1.

### Experimental Process:

### 1. Preparation of Medium:

1) 7.4 g of brain heart infusion powder was weighed and added into a 250 mL conical flask, added with 180 mL of sterile water, fully dissolved and mixed well, followed by moist heat sterilization at high temperature and high pressure, wherein the sterilization conditions were: 121° C., 20 min; the sterilized medium was cooled to room temperature, added with 20 mL (V/V, a final concentration of 10%) of newborn calf serum, mixed well, then aliquoted, and put into a 4° C. refrigerator for later use.

### 2. Strain Resuscitation:

1) A frozen solution stored at −80° C. was taken out, and thawed at room temperature; 100 μL of bacterial solution was taken with a pipette, coated onto a Columbia blood agar plate (finished-product medium), put invertedly in a 2.5 μL culture box, placed in a micro-aerobic gas production bag to achieve a micro-aerobic environment, and cultured at 37° C. for 2-3 days.
2) A sterile inoculation ring was used to pick Helicobacter pylori single colonies (transparent water droplets) grown on the Colombian blood agar medium and inoculated in the above liquid medium; and cultured at 37° C. in a micro-aerobic environment for 3 days.
3) A required concentration (absorbance) for the experiment was reached after culture, and the bacterial solution was taken for subsequent experiments.

### 3. Preparation and Dilution of Compound Mother Solution:

A preparation method of the bile acid-metronidazole conjugate was the same as that of Example 1.

### 4. MIC Determination Using Broth Microdilution Method:

The determination method was the same as that of Example 1.

### (III) Results

The drug concentration at which bacteria ceased to grow or grew at the most significant reduction level was referred to as the MIC value.

TABLE 2
The MIC values of the compounds of the present application for
inhibiting the in-vitro activity of Helicobacter pylori (in μg/mL)

| | MIC (μg/mL) | | | |
|---|---|---|---|---|
| | H. pylori | H. pylori | H. pylori | H. pylori |
| | ATCC | ATCC | NCTC | Sydney strain |
| Compounds | 43504(R) | 26695(S) | 11637(R) | SS1 |
| Metronidazole | >128 | 4 | >128 | 8 |
| | (64-256)^{a} | (1-8)^{b} | (>128)^{c} | |
| UDCA | >128 | >32 | >128 | >32 |
| SCUT1-1 | >128 | 8 | 16 | 8 |
| SCUT1-2 | 8 | 4 | 16 | 8 |
| SCUT1-3 | 4 | 4 | 8 | 8 |
| SCUT1-4 | 4 | 4 | 4 | 4 |
| SCUT1-5 | 4 | 8 | 32 | 16 |
| SCUT1-6 | 8 | 4 | 8 | 16 |
| SCUT1-7 | 8 | 4 | 8 | 8 |
| SCUT1-8 | 16 | 8 | 64 | 8 |

Metronidazole: MIC ≤ 8 μg/mL→Susceptible; MIC > 8 μg/mL→Resistant.
^{a},^{b}, and ^{c}were the MIC values of metronidazole corresponding to this strain reported in related literatures.

It could be seen from Table 2 that the bile acid-metronidazole conjugate showed better inhibitory activities against all four types of Helicobacter pylori, maintained the inhibitory activities of metronidazole for two sensitive strains, and showed strong inhibitory effects on the two metronidazole-resistant strains, ATCC 43504 and NCTC 11637, which obviously reversed drug resistance. According to the dosage of bile acid, the inhibitory activity of bile acid-metronidazole was significantly higher than that of bile acid. Similarly, for the bile acid-metronidazole conjugate, according to the dosage of metronidazole, the inhibitory activity of metronidazole conjugated to bile acid was significantly higher than that of metronidazole itself, and the inhibitory activities of some compounds (e.g., SCUT1-3 and SCUT1-4) against two drug-resistant bacteria were significantly promoted. In summary, under the condition that the inhibitory activities of metronidazole against sensitive strains were maintained, the inhibitory activities of most of the compounds against drug-resistant bacteria were significantly enhanced, which could well inhibit the growth of Helicobacter pylori.

### Experimental Example 3: Evaluation of the In-Vivo Activity of Bile Acid-Metronidazole Conjugate Against Clostridioides difficile Infection

1) Experimental animals: C57BL/6, 4-6 weeks old, female.

### 2) Experimental Process:

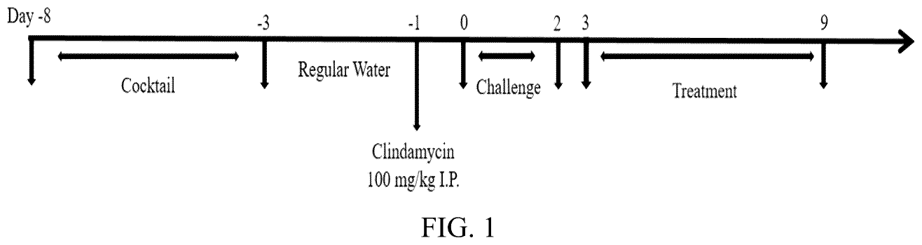

In order to evaluate the efficacy of the compounds of the present application on Clostridioides difficile infection, an animal model of Clostridioides difficile infection was established, as shown in FIG. 1. Mice were administrated with antibiotic cocktail (kanamycin 0.4 mg/mL; gentamicin 0.035 mg/mL; colistin 0.042 mg/mL; metronidazole 0.215 mg/mL and vancomycin 0.045 mg/mL) for five consecutive days (−8 to −3 d) by drinking water, which was then replaced with sterile water; two days after free drinking, the mice were intraperitoneally injected with clindamycin (100 mg/kg), followed by spores (027 Type) challenge 24 h later; and the control group was administrated with normal saline by gavage. On Day 0, the mice were administrated with 10⁵ CFU/mL of spores by gavage; and on Days 1 and 2, the mice were administrated with 10⁶ CFU/mL of spores by gavage. Starting from Day 3, the mice were given 7 days of drug treatment, and all mice were euthanized at the end of the experiment, except for the death of mice during the treatment.

### 3) Experimental Grouping:

Two of the conjugates, SCUT1-2 and SCUT1-6, with the optional in-vitro inhibitory activities were selected. Grouping was performed according to Table 3 by using metronidazole, UDCA and a mixture thereof as controls. The therapeutic effect of the conjugate of the present application in the treatment of Clostridioides difficile infection was evaluated.

TABLE 3
Animal model grouping

| | | | | | Mixture group | | |
|---|---|---|---|---|---|---|---|
| | | | | | (metronidazole + | SCUT1- | SCUT1- |
| | Control | Model | Metronidazole | Ursodeoxycholic | ursodeoxycholic | 2 | 6 |
| Groups | group | group | 50 mg/kg | acid 50 mg/kg | acid) 50 mg/kg | 50 mg/kg | 50 mg/kg |
| Number of | 10 | 10 | 10 | 10 | 10 | 10 | 10 |
| animals | | | | | | | |

### 4) Experimental Results:

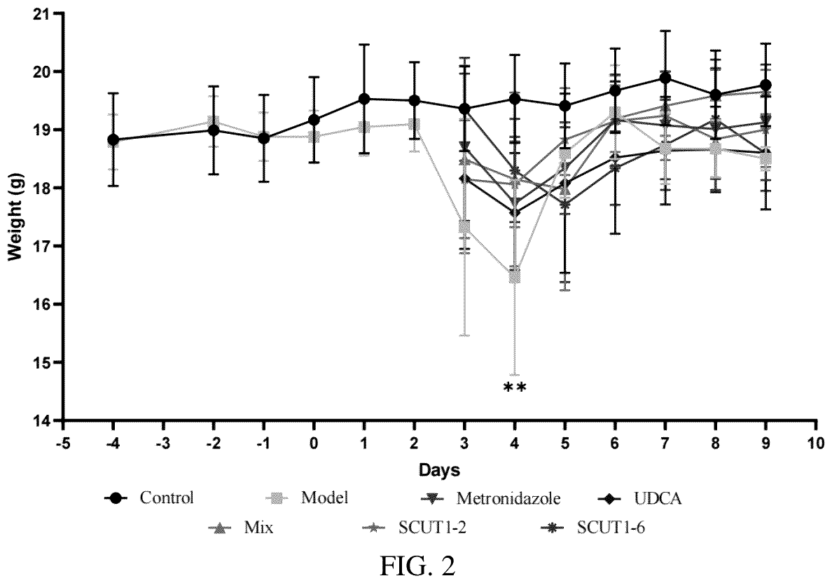

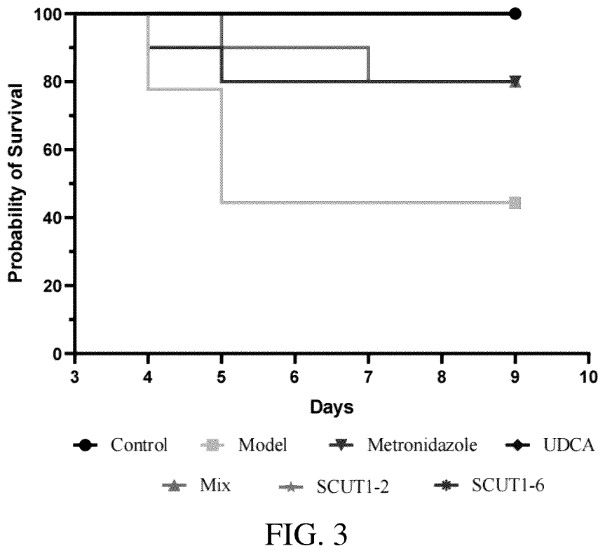

The mice began to show signs of Clostridioides difficile infection from Day 1 and began to show weight loss on Day 2, as shown in FIG. 2. The body weights of the mice in the Model group continued to decrease on Day 3, and reached the lowest value on Day 4, which was significantly different from that of the Control group. The body weights of the mice in each treatment group continued to decrease significantly in the first two days of drug treatment (Day 3 and Day 4), the body weights of the mice in the conjugate SCUT1-6 group and the mixture Mix group continued to decrease until the third day of drug treatment (Day 5), and the body weights of the mice in the other treatment groups gradually increased and tended to be stable after two days of drug treatment. The probability of survival of the mice in the Model group was 44.44%, the probability of survival of the mice in the Metronidazole group and the MIX group (Metronidazole+UDCA) was 80% after drug treatment, and the probability of survival of the mice in the other treatment groups was 100% as no mice died during drug treatment, as shown in FIG. 3.

In FIG. 2, * represented a statistical difference with the Control group *, P<0.05; **, P<0.01.

The diarrhea conditions of the mice before and after treatment were shown in Table 4. Before the administration on Day 3, the diarrhea rate of the mice except the Control group reached 86.67%, as shown in Table 4. The diarrhea rate of the mice in the Model group reached 100% on Day 4, Day 5 and Day 9, and 4 mice died during 7 days of drug treatment (FIG. 3). The diarrhea rate of the mice in each treatment group reached a peak on Day 3 and Day 4, then gradually decreased and tended to be stable. At the endpoint of treatment, the diarrhea rate was 100% in the Model group, 10% in the conjugate SCUT1-2 group, and 25%-50% in the other treatment groups.

TABLE 4
Diarrhea conditions of mice (number of mice)

| | Number of days | | | | | | |
|---|---|---|---|---|---|---|---|
| Groups | 3 | 4 | 5 | 6 | 7 | 8 | 9 |
| Control | 0 (10) | 0 (10) | 0 (10) | 0 (10) | 0 (10) | 0 (10) | 0 (10) |
| Model | 7 (9) | 7 (7) | 4 (4) | 3 (4) | 3 (4) | 3 (4) | 4 (4) |
| Metronidazole | 9 (10) | 8 (9) | 5 (8) | 3 (8) | 3 (8) | 3 (8) | 3 (8) |
| UDCA | 9 (10) | 9 (10) | 7 (10) | 6 (10) | 4 (10) | 5 (10) | 5 (10) |
| Mix | 9 (10) | 8 (9) | 8 (9) | 5 (8) | 3 (8) | 2 (8) | 2 (8) |
| SCUT 1-2 | 10 (10) | 7 (10) | 4 (10) | 2 (10) | 1 (10) | 1 (10) | 1 (10) |
| SCUT1-6 | 8 (10) | 10 (10) | 8 (10) | 7 (10) | 5 (10) | 6 (10) | 5 (10) |

Note:
the number outside the parentheses was the number of mice with diarrhea in this group on the day; and the number inside the parentheses was the number of surviving mice in this group on that day.

Compared with the positive drug Metronidazole group, the UDCA group and the MIX group (Metronidazole+UDCA), the conjugate SCUT1-2 showed a good therapeutic effect. During the treatment, no mice died in the SCUT1-2 group (50 mg/kg), and the diarrhea rate of the mice in this group was 10% at the end of the experiment, which significantly improved the symptoms of diarrhea and weight loss of the mice caused by CDI.

In summary, the bile acid-metronidazole conjugate provided by the present application could effectively inhibit the growth of Clostridioides difficile (ATCC 43255), Bacteroides fragilis (ATCC 25285), Porphyromonas gingivalis (ATCC 33277), Helicobacter pylori and other bacteria, had significant antibacterial activity, achieved a good therapeutic effect in the treatment of Clostridioides difficile infection, and thus had a good application prospect in the field of biomedicine.

The above are only specific embodiments of the present application, but the protection scope of the present application is not limited thereto. Within the technical scope disclosed in the present application, any changes or replacements easily derived by a person skilled in the art shall fall within the protection scope of the present application. Therefore, the protection scope of the present application should be subject to the protection scope of the attached claims.

1. A bile acid-metronidazole conjugate, having a chemical structure as shown in formula (I), or a stereoisomer thereof, or a salt thereof, or a solvate thereof, or a prodrug thereof, or a metabolite thereof; 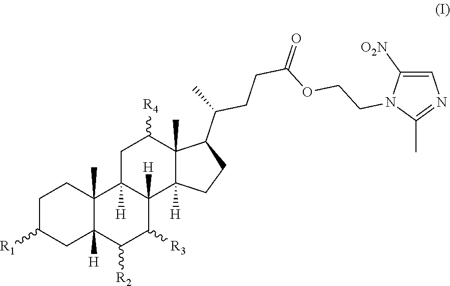
wherein, [symbol] represents a single bond or double bonds, and [symbol] R1, [symbol] R2, [symbol] R3, [symbol] R4 each represents one of —H, —OH, and ═O.

2. The bile acid-metronidazole conjugate according to claim 1, wherein the conjugate, or the stereoisomer thereof, or the salt thereof, or the solvate thereof, or the prodrug thereof, or the metabolite thereof is selected from the following structures:

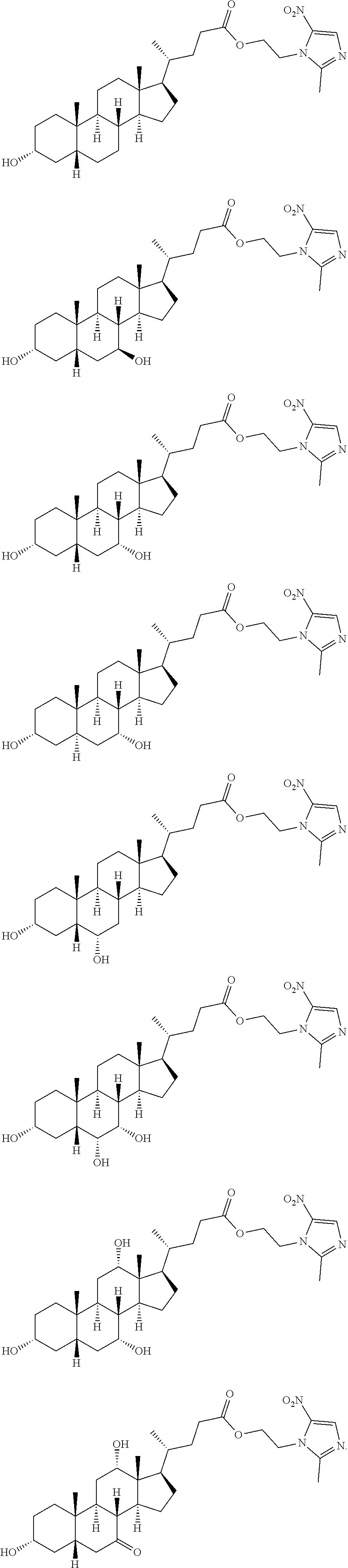

3. The bile acid-metronidazole conjugate according to claim 1, wherein the bile acid-metronidazole conjugate is prepared by an esterification reaction of a bile acid compound with metronidazole.

4. The bile acid-metronidazole conjugate according to claim 3, wherein the bile acid is selected from one of cholic acid, ursodeoxycholic acid, chenodeoxycholic acid, allo-chenodeoxycholic acid, hyodeoxycholic acid, hyocholic acid, lithocholic acid and derivatives thereof.

5. Use of a bile acid-metronidazole conjugate, being use of the bile acid-metronidazole conjugate according to claim 1 in inhibiting the growth of anaerobic bacteria, and in the treatment of diseases caused by infections of the anaerobic bacteria.

6. The use of a bile acid-metronidazole conjugate according to claim 5,
wherein the anaerobic bacteria comprise, but are not limited to, Clostridioides difficile, Helicobacter pylori, Bacteroides fragilis, and Porphyromonas gingivalis.

7. Use of a bile acid-metronidazole conjugate, being use of the bile acid-metronidazole conjugate according to claim 2 in inhibiting the growth of anaerobic bacteria, and in the treatment of diseases caused by infections of the anaerobic bacteria.

8. The use of a bile acid-metronidazole conjugate according to claim 7,
wherein the anaerobic bacteria comprise, but are not limited to, Clostridioides difficile, Helicobacter pylori, Bacteroides fragilis, and Porphyromonas gingivalis.

9. Use of a bile acid-metronidazole conjugate, being use of the bile acid-metronidazole conjugate according to claim 3 in inhibiting the growth of anaerobic bacteria, and in the treatment of diseases caused by infections of the anaerobic bacteria.

10. The use of a bile acid-metronidazole conjugate according to claim 9, wherein the anaerobic bacteria comprise, but are not limited to, Clostridioides difficile, Helicobacter pylori, Bacteroides fragilis, and Porphyromonas gingivalis.

11. Use of a bile acid-metronidazole conjugate, being use of the bile acid-metronidazole conjugate according to claim 4 in inhibiting the growth of anaerobic bacteria, and in the treatment of diseases caused by infections of the anaerobic bacteria.

12. The use of a bile acid-metronidazole conjugate according to claim 11, wherein the anaerobic bacteria comprise, but are not limited to, Clostridioides difficile, Helicobacter pylori, Bacteroides fragilis, and Porphyromonas gingivalis.
